(19) United States
(12) Patent Application Publication
(10) Pub. No.: US 2026/0360555 A1
(43) Pub. Date: Nov. 12, 2026

(54) METHODS AND SYSTEMS TO IDENTIFY FALSE POSITIVE PACS AND PVCS IN ECG DATA

(71) Applicant: KONINKLIJKE PHILIPS N.V., Eindhoven (NL)

(72) Inventors: Joshua Michael Burns, San Diego, CA (US); Justin Tsoneshane Lee, San Diego, CA (US)

(21) Appl. No.: 19/654,623

(22) Filed: Apr. 22, 2026

Related U.S. Application Data
(60) Provisional application No. 63/800,485, filed on May 6, 2025.

(51) Int. Cl. G16H 50/30; A61B 5/00; A61B 5/355; A61B 5/366; G06N 20/00; G16H 15/00; G16H 50/70

(52) U.S. Cl. CPC G16H 50/30; A61B 5/355; A61B 5/366; A61B 5/7221; G06N 20/00; G16H 15/00; G16H 50/70

(57) ABSTRACT

A method for analyzing electrocardiogram (ECG) data, comprising: receiving ECG data for a subject for a first time period; identifying a first window of ECG data within the received ECG data; classifying each of the QRS complexes within the first window of ECG data; identifying each of a plurality of the classified QRS complexes as being a premature atrial contraction (PAC) or a premature ventricular contraction (PVC); analyzing the classified QRS complexes within the first window of ECG data to generate an output comprising an identification of one or more of the classified QRS complexes as being incorrectly identified as a PAC or PVC; removing the QRS complexes identified as being falsely classified as a PAC or PVC; and enumerating a number of remaining QRS complexes identified as a PAC or PVC in the first window or in the received ECG data as a whole.

### CROSS-REFERENCE TO RELATED APPLICATIONS

This patent application claims the priority benefit under 35 U.S.C. § 119(e) of U.S. Provisional Application No. 63/800,485 filed on May 6, 2025, the contents of which are herein incorporated by reference.

### FIELD OF THE DISCLOSURE

The disclosed subject matter generally pertains to methods and symptoms for analyzing ECG data using an ECG analysis system.

### BACKGROUND

Electrocardiogram (ECG) is a non-invasive test that measures the electrical activity of the heart by placing electrodes on the chest and limbs. It is a vital diagnostic tool in cardiology, used to identify a wide range of cardiac conditions, including heart attack, arrhythmia, and heart failure. ECG can also be used to monitor the effectiveness of treatment and detect early signs of complications.

Healthcare professionals will review a patient's ECG data for diagnosis. Although many algorithms exist to facilitate this review, including algorithms that summarize ECG data for a given time period, or provide recommendations or possible diagnoses for the patient, a healthcare professional is typically always involved in a manual review of the patient's ECG data.

However, it is not possible to have medical personal to review the entirety of a subject's ECG records, whether for an in-hospital or ambulatory setting, because the length of the recordings can be multiple days or weeks long. Algorithms that classify ECG data as QRS complexes or arrhythmias are often used to screen these extensive ECG recordings and to present findings for review. Rule-based and deep learning algorithms classify and annotate QRS complexes based on morphology and rhythm to estimate where they originate. These algorithms may not classify QRS complexes as a QRS complex, or may classify QRS complexes incorrectly, which can increase the burden on clinical staff. For example, QRS complexes can be falsely identified or classified as a premature atrial contraction (PAC) or premature ventricular contraction (PVC), which can significantly impact the analysis, interpretation, and diagnosis by a clinician when based in whole or in part on the ECG data.

Accordingly, there is a continued need for methods and systems that facilitate the healthcare professional's review of a patient's ECG data. For example, there is a continued need for methods and systems that more correctly identify or classify PACs and/or PVCs.

### SUMMARY OF THE DISCLOSURE

Thus, there is a continued need to determine and provide more accurate identification and summary of PACs and/or PVCs in a subject's ECG data, which in turn improves clinician analysis and interpretation of the subject's ECG data, and thus significantly improves patient care and outcomes.

Various embodiments and implementations are directed to methods and systems for analyzing ECG data. An ECG analysis system receives ECG data for a subject, and identifies a first window within that data. The QRS complexes within the first window of ECG data are classified as a T-wave, a premature atrial contraction (PAC), a premature ventricular contraction (PVC), or other beat. The classified QRS complexes are then analyzed to identify premature PACs and/or PVCs. A trained machine learning algorithm is used to identify one or more of the classified QRS complexes as being falsely identified as a PAC and/or PVC. These falsely classified PACs and/or PVCs are removed from the ECG data, and the system enumerates the number of remaining QRS complexes identified as a PAC and/or PVC.

According to an aspect, a method for analyzing electrocardiogram (ECG) data is provided. The method includes: receiving ECG data for a subject for a first time period, the ECG data comprise a plurality of cardiac cycles; identifying a first window of ECG data within the received ECG data, wherein the window is a subset of the received ECG data; classifying each of the QRS complexes within the first window of ECG data, wherein classifying comprises classifying each QRS complex as a T-wave, a PAC, a PVC, or other type of heartbeat; identifying each of a plurality of the classified QRS complexes as being a PAC or a PVC; analyzing, using a trained machine learning algorithm, the classified QRS complexes within the first window of ECG data to generate an output, the output comprising an identification of one or more of the classified QRS complexes as being falsely identified as a PAC or PVC; removing, from the first window, the QRS complexes identified as being falsely classified as a PAC or PVC; and enumerating, after said removing, a number of remaining QRS complexes identified as a PAC or PVC in the first window or in the received ECG data as a whole.

According to an embodiment, the ECG data for the subject is identified by identifying ECG data comprising a high PAC or PVC burden.

According to an embodiment, classifying the QRS complexes within the first window of ECG data comprises classifying each QRS complex using a clustering process.

According to an embodiment, the first window comprises an amount of ECG data that cannot be analyzed by a human being in a medically-relevant timeframe.

According to an embodiment, the method further includes generating and providing, via a user interface, a report comprising the enumeration of the number of remaining QRS complexes identified as a PAC or PVC.

According to an embodiment, identifying each of a plurality of the classified QRS complexes as being a PAC or PVC comprises an algorithm configured to identify PACs and/or PVCs.

According to an embodiment, the method further includes training the machine learning algorithm, comprising the steps of: receiving training data comprising ECG data with identified PACs or PVCs for a plurality of ECG studies for a plurality of patients; training the machine learning algorithm, with the received training data, to identify PACs and/or PVCs in ECG data; and storing the trained machine learning algorithm.

In accordance with another aspect is a system for analyzing electrocardiogram (ECG) data. The system includes: ECG data for a subject for a first time period, the ECG data comprise a plurality of cardiac cycles; a machine learning algorithm trained to identify premature atrial contractions (PACs) or premature ventricular contractions (PVCs) in ECG data; a processor configured to: (i) identify a first window of ECG data within the received ECG data, wherein the window is a subset of the received ECG data; (ii) classify each of the QRS complexes within the first window of ECG data, wherein classifying comprises classifying each QRS complex as a T-wave, a PAC, a PVC, or other type of heartbeat; (iii) identify each of a plurality of the classified QRS complexes as being a PAC or PVC; (iv) analyze, using the trained machine learning algorithm, the classified QRS complexes within the first window of ECG data to generate an output, the output comprising an identification of one or more of the classified QRS complexes as being falsely identified as a PAC or PVC; (v) remove, from the first window, the QRS complexes identified as being falsely classified as a PAC or PVC; and (vi) enumerate, after said removing, a number of remaining QRS complexes identified as a PAC or PVC in the first window or in the received ECG data as a whole.

According to an embodiment, the processor is further configured to train the machine learning algorithm, comprising: receiving training data comprising ECG data with identified PACs or PVCs for a plurality of ECG studies for a plurality of patients; training the machine learning algorithm, with the received training data, to identify PACs or PVCs in ECG data; and storing the trained machine learning algorithm.

According to an embodiment, the system includes an ECG device configured to obtain the ECG data.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded with a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a flowchart of a method for analyzing electrocardiogram (ECG) data, in accordance with an embodiment.

FIG. 2 is a schematic representation of an ECG analysis system, in accordance with an embodiment.

FIG. 3 is a schematic representation of an ECG environment, in accordance with an embodiment.

FIG. 4 is a flowchart of a method for training a PAC or PVC identification algorithm, in accordance with an embodiment.

FIG. 5 is a graph of PVC burden reduction using the methods and systems described or otherwise envisioned herein, in accordance with an embodiment.

FIG. 6 is a graph of PVC burden reduction using the methods and systems described or otherwise envisioned herein, in accordance with an embodiment.

FIG. 7 is a graph of PVC burden reduction using the methods and systems described or otherwise envisioned herein, in accordance with an embodiment.

FIG. 8 is a graph of PVC burden reduction using the methods and systems described or otherwise envisioned herein, in accordance with an embodiment.

FIG. 9 is a graph of PVC burden reduction using the methods and systems described or otherwise envisioned herein, in accordance with an embodiment.

FIG. 10 is a graph of PVC burden reduction using the methods and systems described or otherwise envisioned herein, in accordance with an embodiment.

FIG. 11 is a graph of PVC burden reduction using the methods and systems described or otherwise envisioned herein, in accordance with an embodiment.

FIG. 12 is a graph of PVC burden reduction using the methods and systems described or otherwise envisioned herein, in accordance with an embodiment.

### DETAILED DESCRIPTION OF EMBODIMENTS

The present disclosure describes various embodiments of a system and method configured for analyzing ECG data. More generally, Applicant has recognized and appreciated that it would be beneficial to provide methods and systems for improved ECG analysis of a subject. An ECG analysis system receives ECG data for a subject, and identifies a first window within that data. The QRS complexes within the first window of ECG data are classified as a T-wave, a PAC, a PVC, or other beat. The classified QRS complexes are then analyzed to identify PACs or PVCs. A trained machine learning algorithm is used to identify one or more of the classified QRS complexes as being falsely identified as a PAC or PVC. These falsely classified PACs or PVCs are removed from the ECG data, and the system enumerates the number of remaining QRS complexes identified as a PAC or PVC.

According to an embodiment, the systems and methods described or otherwise envisioned herein can, in some non-limiting embodiments, be implemented as a component of or an extension of ECG systems. For example, one application of the embodiments and implementations herein is to improve systems such as, e.g., the Philips® ECG and/or IntelliSpace® line of diagnostic and reporting tools (manufactured by Koninklijke Philips, N.V.), among many other products. However, the disclosure is not limited to these devices or systems, and thus the disclosure and embodiments disclosed herein can encompass any ECG method, device, or system.

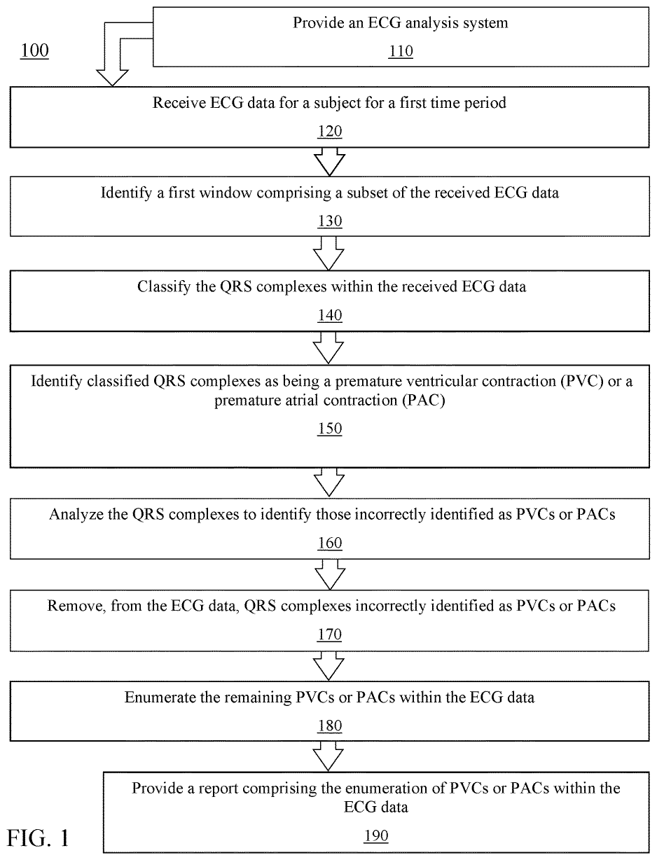

Referring to FIG. 1, in one embodiment, is a flowchart of a method 100 for analyzing ECG data using an ECG analysis system. The methods described in connection with the figures are provided as examples only, and shall be understood to not limit the scope of the disclosure. The ECG analysis system can be any of the systems described or otherwise envisioned herein. The ECG analysis system can be a single system or multiple different systems.

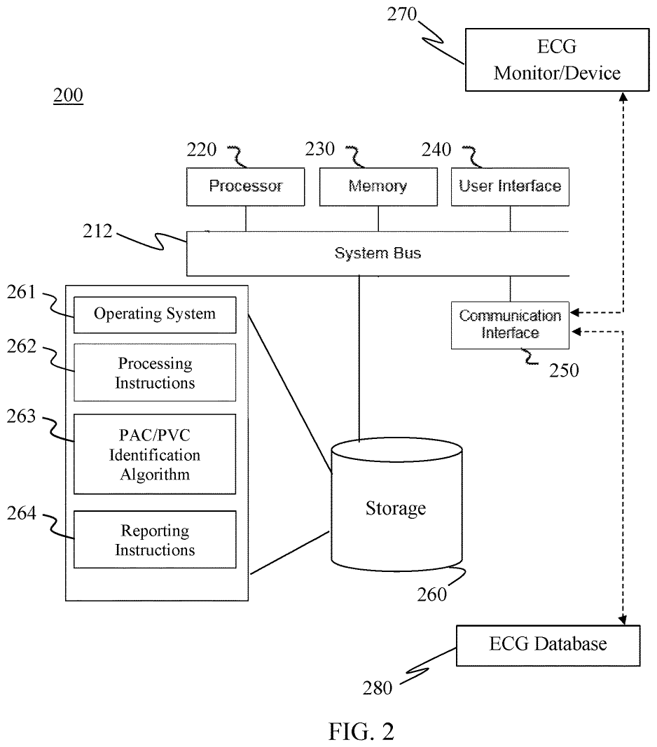

At step 102 of the method, an ECG analysis system 200 is provided. Referring to an embodiment of an ECG analysis system 200 as depicted in FIG. 2, for example, the system comprises one or more of a processor 220, memory 230, user interface 240, communication interface 250, and storage 260, interconnected via one or more system buses 212. It will be understood that FIG. 2 constitutes, in some respects, an abstraction and that the actual organization of the components of the system 200 may be different and more complex than illustrated. Additionally, ECG analysis system 200 can be any of the systems described or otherwise envisioned herein. Other elements and components of ECG analysis system 200 are disclosed and/or envisioned elsewhere herein.

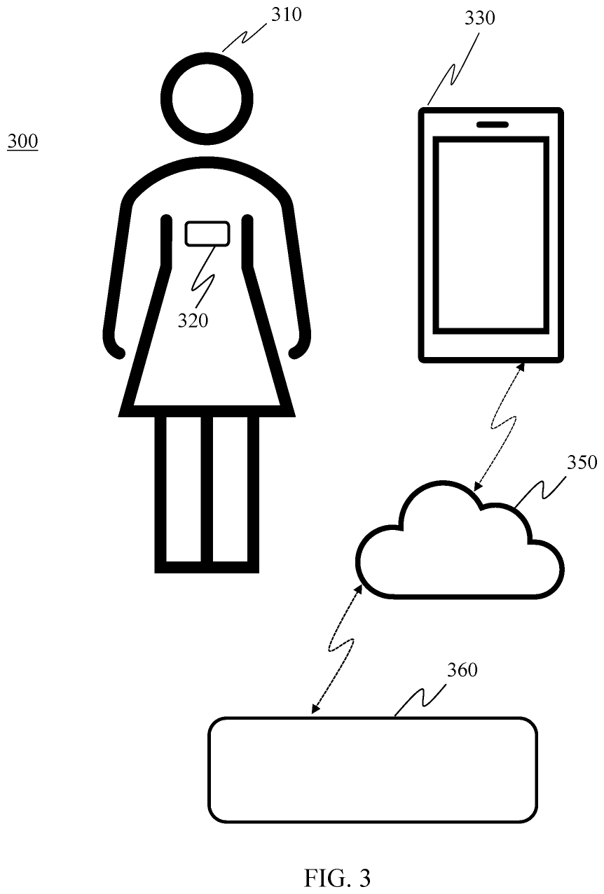

According to an embodiment, the ECG analysis system 200 is or comprises or is in local and/or remote communication with an ECG monitor or device 270. The ECG monitor or device 270 can be any ECG monitor or device, including but not limited to an ambulatory ECG monitor or device. Notably, however, the methods and systems described or otherwise envisioned herein are not limited to ambulatory ECG monitors or devices. According to an embodiment, the ECG monitor or device 270 captures the electrical activity of the subject's heart over a first time period. Referring to FIG. 3, in one embodiment, is an ECG monitoring environment 300. The ECG monitoring environment comprises a subject 310 that is being monitored using an ECG device 320. The environment further comprises a computerized device 330. The computerized device 330 can transmit information, such as ECG data, via a wired and/or wireless communication system 350 to a remote component 360 of the ECG analysis system, such as a database or another application. This transmission of information can be in real-time, in batches, and/or all at once such as when monitoring is complete or the subject or healthcare professional directs or requests the transmission of the information.

In addition or alternatively, according to an embodiment, the ECG analysis system 200 is or comprises or is in local and/or remote communication with an ECG database 280. The ECG database comprises stored ECG data for one or more patients, received from an ECG monitor or device.

Returning to method 100 in FIG. 1, at step 120 of the method, the ECG analysis system 200 receives ECG data for a subject for a first time period. The ECG data will comprise a plurality of cardiac cycles. The first time period can be any time period, such as minutes or hours. For example, according to an embodiment, the first time period is 24 hours, although many other time periods, shorter or longer, are possible. For example, the ECG data may be days or even weeks of data. The ECG data may be received from any source, including local or remote sources. The ECG data may be received from the ECG device 270 or the ECG database 280. Once received, the ECG data may be utilized immediately and/or it may be stored in local and/or remote memory for future use.

At step 130 of the method, the ECG analysis system 200 identifies a first window of ECG data within the received ECG data. The first window will be a subset of the received ECG data. The first window can be any subset of the received ECG data. The size of the first window can be determined by the amount of total received ECG data, a predetermined window size, a learned or experimentally-derived window size, and/or any other mechanism.

According to an embodiment, the ECG analysis system identifies ECG data for a subject, either in whole or as part of identifying the first window of ECG data within the received ECG data, by identifying ECG data comprising a high PAC or PVC burden. PVC burden of ECG data can be determined by analyzing the ECG data with an algorithm that identifies PVCs within ECG data. Similarly, PAC burden of ECG data can be determined by analyzing the ECG data with an algorithm that identifies PACs within ECG data. Thus, the algorithm can provide an output that comprises a PAC or PVC burden or enumeration within the analyzed ECG data. ECG data comprise a “high” PVC burden can be identified based on a predetermined threshold of PVCs (where the threshold is a percentage of PVCs, a number of PVCs per a specific time period such as an hour or the total ECG data, or another threshold that is a learned or experimentally-derived amount of PVCs within the ECG data). ECG data comprise a “high” PAC burden can be identified based on a predetermined threshold of PACs (where the threshold is a percentage of PACs, a number of PACs per a specific time period such as an hour or the total ECG data, or another threshold that is a learned or experimentally-derived amount of PACs within the ECG data). This can prioritize analysis of ECG data by the ECG analysis system that comprises a threshold number of PACs (i.e., a high PAC burden) or PVCs (i.e., a high PVC burden), thereby improving clinician analysis and diagnosis of ECG data and both speeding up and improving patient care.

According to an embodiment, the first window comprises an amount of ECG data that cannot be analyzed by a human being in a medically-relevant timeframe. For example, the ECG data may comprise a day's worth of ECG data (where, if the average heartbeat is 60-100 beats per minute, that is 86,400 to 144,000 heartbeats in a day; if a clinician reviews a day's worth of heartbeats at approximately 1 second/heartbeat, that is approximately 24-40 hours of review). When the ECG data is more than a day, the amount of review becomes further impossible. Further, given the onset of human fatigue, a human cannot process this volume of data accurately.

At step 140 of the method, the QRS complexes within the first window of ECG data are analyzed and classified. According to an embodiment, the QRS complexes within the ECG data are analyzed by a classification algorithm of the ECG analysis system 200, and/or by a classification algorithm of another system. There are known algorithms that classify QRS complexes within ECG data, in order to facilitate additional downstream analysis by a clinician or another algorithm. According to an embodiment, the QRS complexes within the ECG data are classified in any of a variety of different ways. For example, the QRS complexes can be classified as a T-wave, a PAC, a PVC, or other beat. Many other forms and categories of classification are possible. According to one embodiment, analyzing the ECG data by the classification algorithm includes identifying QRS complexes within the ECG data, prior to grouping the QRS complexes.

According to an embodiment, the QRS complexes within the ECG data are classified using a clustering process, where the classification algorithm groups the QRS complexes into clusters based on their similarities. Common clustering methods include K-means, hierarchical clustering, and density-based clustering. Once clustered, the algorithm can label each cluster. For example, one cluster might represent normal beats, while another might represent PVCs or other types of abnormal beats.

Thus, the output of step 140 may be the classification of the QRS complexes in the ECG data into two or more different categories of complexes. Once classified, the ECG data may be utilized immediately and/or it may be stored in local and/or remote memory for future use. If the classification is performed by a classification algorithm of another system, the output of the classification can be sent to the ECG analysis system 200, or otherwise received by the system.

At step 150 of the method, the classified QRS complexes are analyzed to identify premature atrial contractions (PACs) and/or premature ventricular contractions (PVCs). This may be performed simultaneously with the classification process, or after the classification process, of step 140. For example, the classification algorithm can identify, group, and/or classify the QRS complexes, and can thus identify classified QRS clusters as PACs or PVCs as part of that process. Alternatively, the classified QRS complexes can be analyzed by another algorithm that is trained or designed to identify PACs or PVCs within ECG data. This algorithm can be an algorithm of the ECG analysis system 200, or it can be an algorithm of another system. PACs or PVCs can be identified within ECG data using the algorithm according to known methods for PAC or PVC identification.

Thus, the output of step 150 may be an identification of PACs and/or PVCs within the received ECG data, where identification is an enumeration of PACs or PVCs within the received ECG data for a subject (such as a total number, a percentage, a number of PVCs per time period such as an hour or minute, and/or any other quantification of PACs or PVCs). Once the PACs or PVCs are identified, the resulting ECG data may be utilized immediately and/or it may be stored in local and/or remote memory for future use. If the identification is performed by an identification algorithm of another system, the output of the identification can be sent to the ECG analysis system 200, or otherwise received by the system.

At step 160 of the method, the classified/identified ECG data is analyzed to identify classified QRS complexes that were falsely identified as a PAC or PVC. According to an embodiment, the ECG data is analyzed by a trained machine learning algorithm in order to generate an output, where the output is an identification of one or more of the classified QRS complexes as being falsely identified as a PAC or PVC (where “falsely” means incorrectly or inaccurately identified as being a PAC or PVC, i.e., a false positive). Thus, the trained machine learning algorithm, which can be identified as a PAC/PVC identification algorithm, is trained to analyze the input—the classified ECG data comprising the initial identification of PACs or PVCs—in order to generate the output, namely identification of one or more of the classified QRS complexes that were incorrectly identified as a PAC or PVC.

The trained PAC/PVC identification algorithm can be any model that can be trained to utilize the input to generate the output, as described or otherwise envisioned herein. For example, the trained PAC/PVC identification algorithm can be a neural network or other trained machine learning model. Thus, according to an embodiment, the ECG analysis system comprises a trained PAC/PVC identification algorithm that receives the input data and generates the outputs data as described or otherwise envisioned herein.

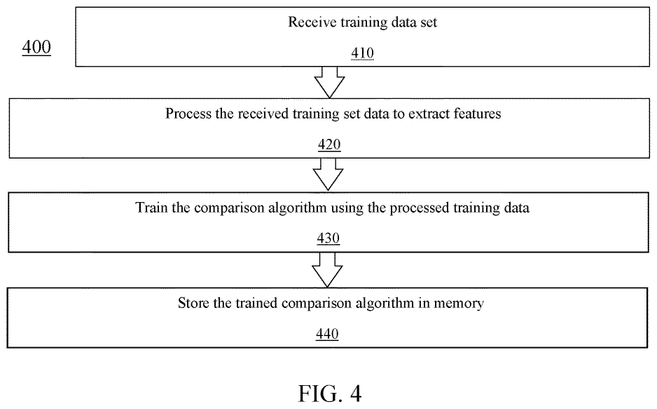

The PAC/PVC identification algorithm can be trained in a variety of different ways. According to one embodiment, the PAC/PVC identification algorithm is trained in an unsupervised manner or in a supervised manner. Referring to FIG. 4, in one embodiment, is a flowchart of a method 400 for training the PAC/PVC identification algorithm of the ECG analysis system 200. This method may be performed by the ECG analysis system or may be performed by another system such as a specialized machine learning model training system.

At step 410 of the method, the training system receives training data which will be used to train the model. The training data can be any data sufficient to train the model to utilize the described input data to generate the described output. For example, the training data may comprise ECG data with identified PACs and/or PVCs for a plurality of ECG studies for a plurality of patients, including with ground truth optimization. This training data, which could be utilized in a supervised or unsupervised manner, can comprise 100s or 1000s of ECG studies, and can optionally be updated with new studies. The training data may also comprise other information. This training data may be obtained and curated by an expert such as a clinician, or it may be obtained and curated under the supervision of a clinician, or it may be obtained and utilized without curation. The training data may be received from any source. For example, the training data may be received from an electronic medical record database or system, or any other component of the ECG analysis system or a training system. According to an embodiment, the ECG analysis system 200 comprises or is in direct or indirect communication with a database which comprises some or all of the training data set.

According to an embodiment, the training system may comprise a data pre-processor or similar component or algorithm configured to process the received training data. For example, the data pre-processor analyzes the training data to remove noise, bias, errors, and other potential issues. The data pre-processor may also analyze the input data to remove low quality data. Many other forms of data pre-processing or data point identification and/or extraction are possible.

At step 420 of the method, the training system trains the PAC/PVC identification algorithm, using the training data, to classify identified PACs and/or PVCs in ECG data as being either: (1) properly identified as a PAC or PVC or (2) incorrectly identified as a PAC or PVC. Other categories are possible, such as (3) unclear or indeterminable, among others. The PAC/PVC identification algorithm is trained using any method for training such a model. The trained PAC/PVC identification algorithm is a unique model based on the training data used to train the model. Following training, the system comprises a trained PAC/PVC identification algorithm.

Thus, following training, the PAC/PVC identification algorithm is a specialized model configured to receive the input and generate the very specific output, namely the identification of PACs or PVCs in the ECG data as being: (1) properly identified as a PAC or PVC or (2) incorrectly identified as a PAC or PVC (among other possible identifications).

At step 430 of the method, the trained PAC/PVC identification algorithm is stored for future use. According to an embodiment, the trained PAC/PVC identification algorithm may be stored in local or remote storage.

### EXAMPLE

Described below is an example of PAC/PVC identification algorithm training using embodiments of the methods and systems described or otherwise envisioned herein. The examples are understood to help explain embodiments, and are understood to be non-limiting examples or explanations of certain embodiments of the methods and systems described or otherwise envisioned herein.

According to an embodiment, ECG records from prescriptions with high PAC and/or PVC burden were extracted for a given time period. A high PAC or PVC burden can be defined as described or otherwise envisioned herein. The given time period can be any time period, as described or otherwise envisioned herein. These ECG records were divided into a training dataset and a testing dataset. Individual beats within each ECG record were labeled as T-wave, a PAC, a PVC, or other using a clustering process in both the training dataset and the testing dataset.

The training dataset was used to train the PAC/PVC identification algorithm as described or otherwise envisioned herein. For example, according to an embodiment, a featurization stage of training generated appropriate features to distinguish T-waves from PACs from PVCs and other artifacts. The training state further tuned thresholds to separate T-waves from other V-annotated fiducial points. A final model was then selected based on accuracy of PAC or PVC identification.

According to an embodiment, in order to test the final model, a generalized test set consisting of ECG records across a period of time along with associated prescription full disclosure data were obtained. The selected model was run against generalized test sets to determine effects of the filter on conditions such as ventricular tachycardia. The model was then deployed.

According to an embodiment, the final PAC/PVC identification algorithm utilizes one or more of the following features as part of the analysis: (1) previous beat and current beat annotation (categorical); (2) current beat and next beat annotation (categorical); (3) previous beat and current beat and next beat annotation (categorical); (4) left RR interval (numeric) (where the RR interval is the time between two consecutive R-waves (heartbeats)); (5) right RR interval (numeric); (6) left RR interval and right RR interval (numeric); (7) log-likelihood of the current beat's RR-interval relative to the current RR-interval distribution (numeric); and/or (8) log-likelihood of the next beat's RR-interval relative to the current RR-interval distribution (numeric). Many other features are possible. Accordingly, since these are features that can be utilized by the PAC/PVC identification algorithm, the system or the algorithm will extract or identify these features in the input data in order for the algorithm to perform the subsequent analysis.

According to an embodiment, the final PAC/PVC identification algorithm comprises one or more of the following: (1) log-likelihood of the current and next beat's RR-interval relative to the current RR-interval distribution is determined by running a median filter to get distribution parameters and modeling the distribution as an Inverse Gaussian; (2) direct annotation-based features are calculated; (3) a feature matrix is generated from derived features associated with V-beat annotations; (4) categorical features are one-hot encoded; (5) missing numeric features are filled in with the median within the dataset; (6) numeric features are scaled using StandardScaler or another algorithm; (7) a feature matrix is passed to a random forest model; (8) and the random forest model returns predictions as to which one of the PVCs are actually oversensed T-waves (which is one example where there is a misclassification of PVCs). According to an embodiment, a random forest model was utilized because it has similar statistics to the closest performing model, SVM, while being much faster to train. However, many other models may be utilized.

Returning to method 100 in FIG. 1, at step 170 of the method, the system removes the QRS complexes incorrectly identified as being a PAC or PVC (i.e., removal of false positives). Removing QRS complexes incorrectly identified as being a PAC or PVC can comprise any action that prevents the QRS complex from being identified or enumerated as a PAC or PVC. Notably, “removal” or “removing” can comprise simply changing a label of the QRS complex, among other actions. Once the PACs or PVCs are removed, the resulting ECG data may be utilized immediately and/or it may be stored in local and/or remote memory for future use.

At step 180 of the method, after the ECG analysis system 200 removes the QRS complexes incorrectly identified as being a PAC or PVC, the system enumerates the remaining QRS complexes identified as a PAC or PVC in the first window or in the received ECG data as a whole. The enumeration can be any identification of the amount of remaining PACs or PVCs within the received ECG data for a subject (such as a total number, a percentage, a number of PACs or PVCs per time period such as an hour or minute, and/or any other quantification of remaining PACs or PVCs). Once the PACs and/or PVCs are enumerated, the resulting information or ECG data may be utilized immediately and/or it may be stored in local and/or remote memory for future use.

At step 190 of the method, the ECG analysis system 200 generates and provides, via a user interface of the system, a report comprising the enumeration of the number of remaining QRS complexes identified as a PAC or PVC. The enumeration can be any quantification or analysis of the remaining PACs or PVCs within the ECG data. The report may also comprise any other information, such as information about the patient, the procedure, or any other information provided or generated as part of the methods and systems described or otherwise envisioned herein. Thus, according to an embodiment, the provided output can be any of the information as described or otherwise envisioned herein. The system may provide the information to a user via any mechanism, including but not limited to a visual display. The information may be communicated by wired and/or wireless communication to another device. For example, the system may communicate the information to a monitor, screen, mobile phone, computer, laptop, wearable device, and/or any other device configured to allow display and/or other communication of the information.

### EXAMPLE(S)

Described below are examples of ECG analysis using embodiments of the methods and systems described or otherwise envisioned herein. The examples are understood to help explain embodiments, and are understood to be non-limiting examples or explanations of certain embodiments of the methods and systems described or otherwise envisioned herein.

According to an embodiment, the methods and systems described or otherwise envisioned herein were tested against ECG strips and patient databases. Referring to TABLES 1, 2, AND 3 is the performance of various machine learning algorithms for training, validation, and test datasets. T and PVC are the detection of over sensed T-waves and true PVCs, respectively.

TABLE 1
Performance of models for training.

| | training set | | | | |
|---|---|---|---|---|---|
| model | T Se | T PPV | T ROC AUC | T PR AUC | PVC Se |
| randomforest | 1.00 | 0.75 | 1.00 | 1.00 | 1.00 |
| svm | 0.81 | 0.73 | 0.89 | — | 0.99 |
| baseline | 0.50 | 0.57 | 0.50 | 0.62 | 0.50 |
| randomforest | 1.00 | 0.75 | 1.00 | 1.00 | 1.00 |
| qda | 0.78 | 0.67 | 0.73 | 0.75 | 0.67 |
| lda | 0.71 | 0.71 | 0.84 | 0.79 | 0.99 |
| naivebayes | 0.74 | 0.70 | 0.81 | 0.77 | 0.90 |
| logisticregression | 0.74 | 0.71 | 0.85 | 0.80 | 0.98 |
| threshold | 1.00 | 0.00 | 0.50 | 0.57 | 0.00 |
| threshold | 0.89 | 0.69 | 0.65 | 0.57 | 0.41 |
| threshold | 0.54 | 0.66 | 0.70 | 0.57 | 0.87 |
| threshold | 1.00 | 0.00 | 0.50 | 0.57 | 0.00 |
| threshold | 0.22 | 0.61 | 0.60 | 0.57 | 0.99 |
| threshold | 1.00 | 0.00 | 0.50 | 0.57 | 0.00 |
| threshold | 1.00 | 0.00 | 0.50 | 0.57 | 0.00 |
| threshold | 1.00 | 0.00 | 0.50 | 0.57 | 0.00 |
| threshold | 1.00 | 0.00 | 0.50 | 0.57 | 0.00 |
| threshold | 1.00 | 0.00 | 0.50 | 0.57 | 0.00 |
| threshold | 1.00 | 0.00 | 0.50 | 0.57 | 0.00 |
| threshold | 0.01 | 0.58 | 0.50 | 0.57 | 1.00 |
| threshold | 0.02 | 0.58 | 0.51 | 0.57 | 1.00 |
| threshold | 0.49 | 0.65 | 0.69 | 0.57 | 0.91 |
| threshold | 0.62 | 0.67 | 0.70 | 0.57 | 0.80 |
| baseline | 0.50 | 0.57 | 0.50 | 0.62 | 0.50 |

TABLE 2
Performance of models for validation.

| | validation set | | | | |
|---|---|---|---|---|---|
| model | T Se | T PPV | T ROC AUC | T PR AUC | PVC Se |
| randomforest | 0.81 | 0.87 | 0.88 | 0.89 | 0.99 |
| svm | 0.81 | 0.87 | 0.88 | — | 0.99 |
| baseline | 0.51 | 0.23 | 0.50 | 0.61 | 0.50 |
| randomforest | 0.81 | 0.87 | 0.88 | 0.89 | 0.99 |
| qda | 0.80 | 0.46 | 0.76 | 0.76 | 0.72 |
| lda | 0.76 | 0.82 | 0.86 | 0.80 | 0.99 |
| naivebayes | 0.74 | 0.70 | 0.82 | 0.79 | 0.94 |
| logisticregression | 0.78 | 0.82 | 0.86 | 0.81 | 0.99 |
| threshold | 1.00 | 0.23 | 0.50 | 0.52 | 0.00 |
| threshold | 0.90 | 0.30 | 0.64 | 0.52 | 0.38 |
| threshold | 0.50 | 0.53 | 0.69 | 0.52 | 0.90 |
| threshold | 1.00 | 0.23 | 0.50 | 0.52 | 0.00 |
| threshold | 0.16 | 0.69 | 0.57 | 0.52 | 0.99 |
| threshold | 1.00 | 0.23 | 0.50 | 0.52 | 0.00 |
| threshold | 1.00 | 0.23 | 0.50 | 0.52 | 0.00 |
| threshold | 1.00 | 0.23 | 0.50 | 0.52 | 0.00 |
| threshold | 1.00 | 0.23 | 0.50 | 0.52 | 0.00 |
| threshold | 1.00 | 0.23 | 0.50 | 0.52 | 0.00 |
| threshold | 1.00 | 0.23 | 0.50 | 0.52 | 0.00 |
| threshold | 0.00 | 0.80 | 0.50 | 0.52 | 1.00 |
| threshold | 0.02 | 0.82 | 0.51 | 0.52 | 1.00 |
| threshold | 0.42 | 0.56 | 0.66 | 0.52 | 0.93 |
| threshold | 0.60 | 0.49 | 0.71 | 0.52 | 0.85 |
| baseline | 0.51 | 0.23 | 0.50 | 0.61 | 0.50 |

TABLE 3
Performance of models for testing.

| | test set | | | | |
|---|---|---|---|---|---|
| model | T Se | T PPV | T ROC AUC | T PR AUC | PVC Se |
| randomforest | 0.82 | 0.91 | 0.89 | 0.92 | 0.99 |
| svm | 0.82 | 0.90 | 0.89 | — | 0.99 |
| baseline | 0.51 | 0.32 | 0.50 | 0.66 | 0.50 |

The model was further tested using prescriptions (i.e., ECG strips from subjects as described or otherwise envisioned herein), where some of the prescriptions had a low PVC burden and some had a high PVC burden. According to an embodiment, the general population of ECG data consisted of strips selected over a period of time, and the annotation full disclosure datasets associated with the strip prescriptions. FIGS. 1 and 2 demonstrate the PVC beat burden/count reduction as a result of the filter.

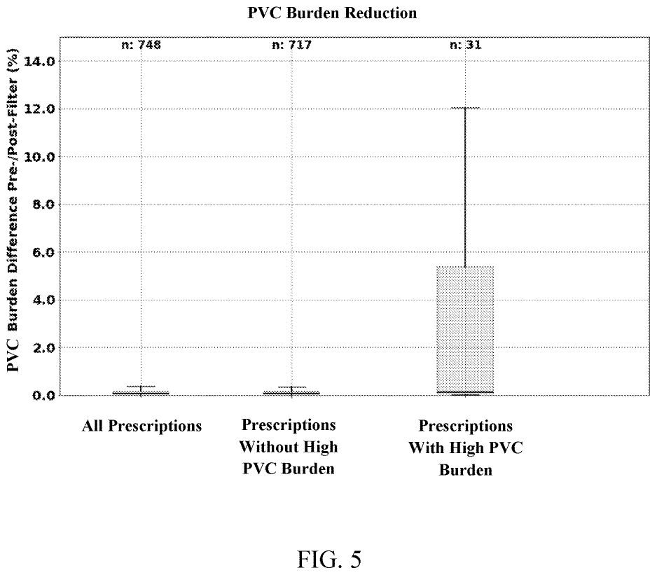

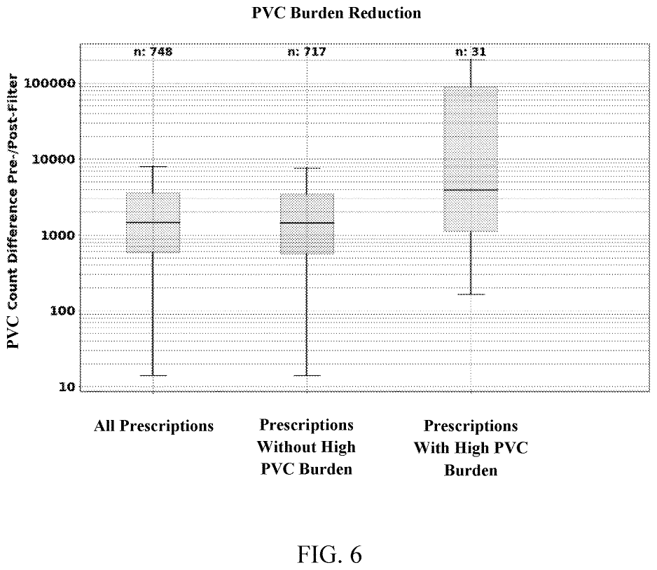

Referring to FIGS. 5 and 6, in accordance with an embodiment, show the PVC beat burden reduction (FIG. 5) and count reduction (FIG. 6) as a result of the methods and systems described or otherwise envisioned herein.

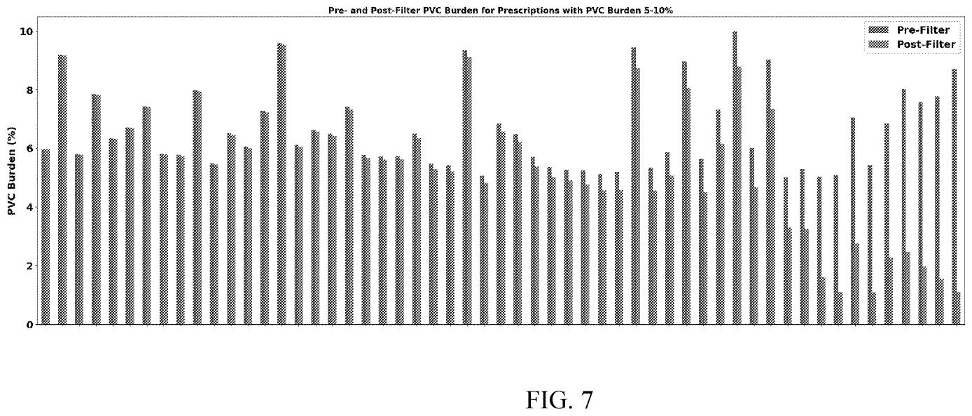

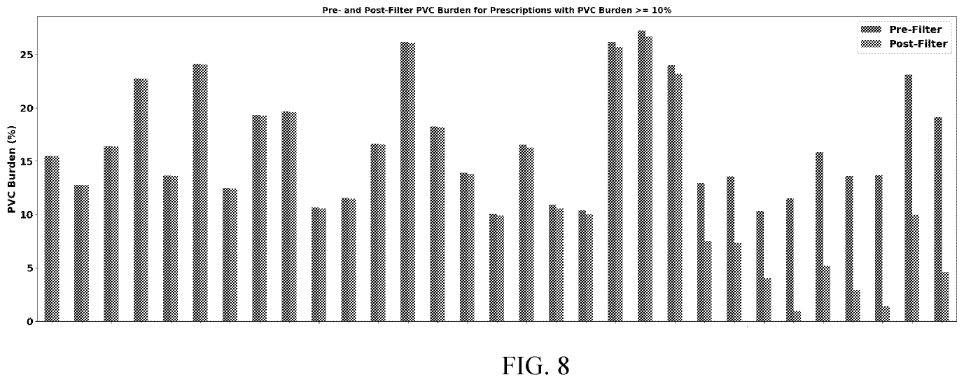

Referring to FIG. 7 (PVC burden 5-10%) and FIG. 8 (PVC burden>10%), in accordance with an embodiment, are PVC burden (pre- and post-filter) by prescription.

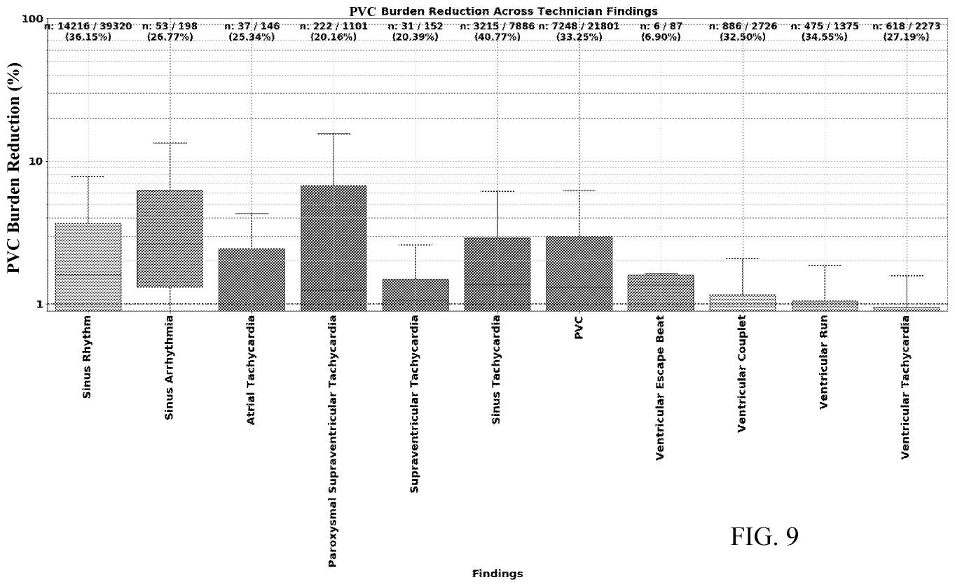

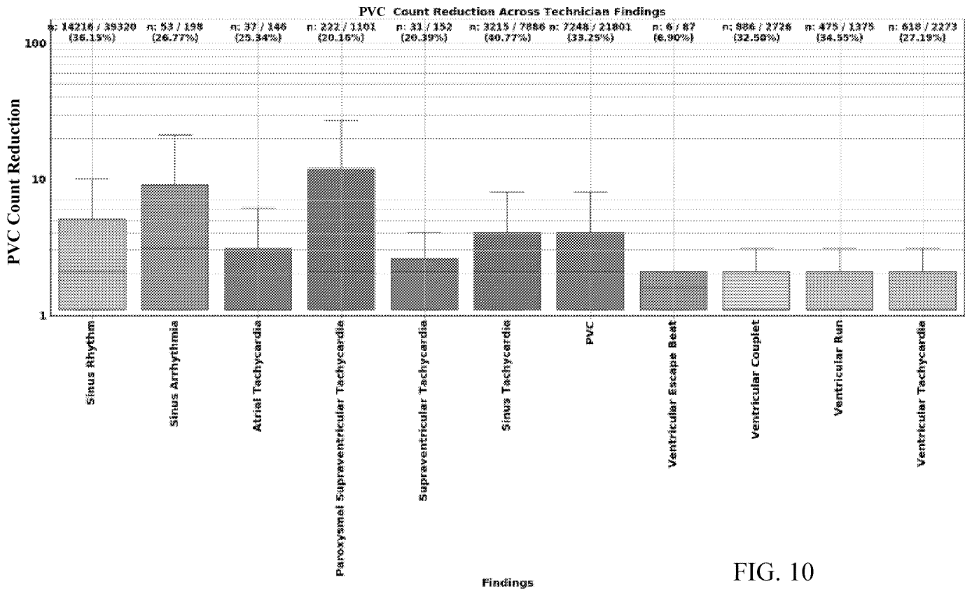

Referring to FIGS. 9 and 10, in accordance with an embodiment, the model shows great performance in removing PVCs where technician findings should not include PVCs and maintaining PVCs in technician findings that include PVCs. It is important to note that there are higher median burden/count reductions in sinus rhythm and sinus arrhythmia findings than in ventricular tachycardia, which has almost no reduction.

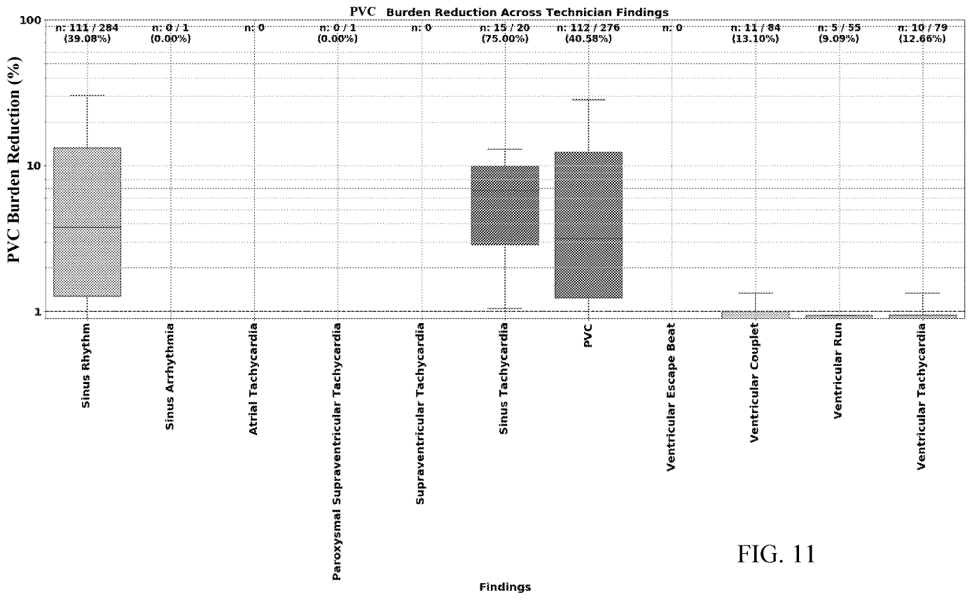

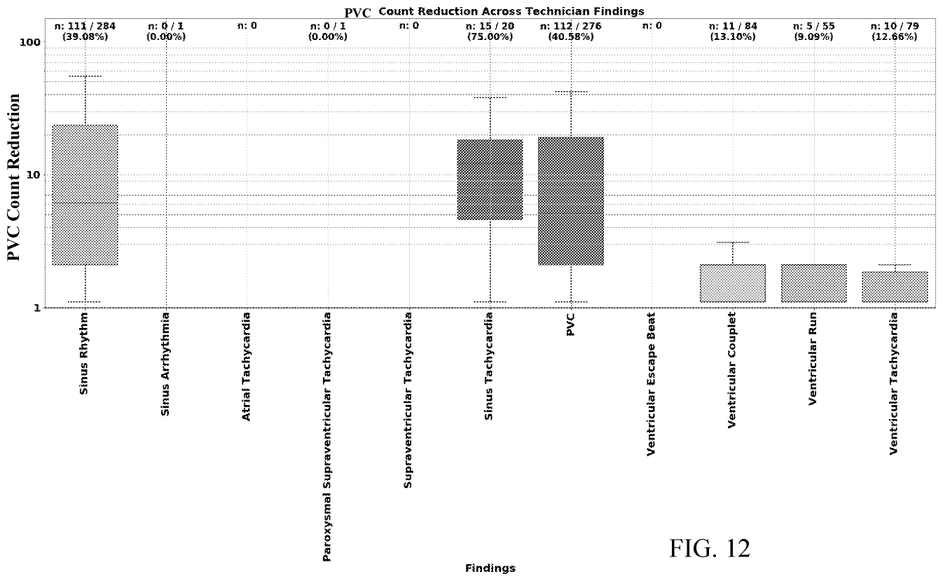

Referring to FIGS. 11 and 12, in accordance with an embodiment, the model shows an improved trend for prescriptions with at least 10% PVC burden. For these high PVC burden prescriptions, there is a dramatic shift in how many PVCs are generally removed across strips. sinus rhythm and sinus tachycardia events have the highest removed, with those PVCs generally either noise or over sensed T-waves. There is PVC reduction in strips with PVC findings, but it is difficult to determine whether the beats being removed are true PVCs or noise.

Referring again to FIG. 2 is a schematic representation of an ECG analysis system 200. System 200 may be any of the devices or systems described or otherwise envisioned herein, and may comprise any of the components described or otherwise envisioned herein. It will be understood that FIG. 2 constitutes, in some respects, an abstraction and that the actual organization of the components of system 200 may be different and more complex than illustrated.

According to an embodiment, system 200 comprises a processor 220 capable of executing instructions stored in memory 230 or storage 260 or otherwise processing data to, for example, perform one or more steps of the method. Processor 220 may be formed of one or multiple modules. Processor 220 may take any suitable form, including but not limited to a microprocessor, microcontroller, multiple microcontrollers, circuitry, field programmable gate array (FPGA), application-specific integrated circuit (ASIC), a single processor, or plural processors.

Memory 230 can take any suitable form, including a non-volatile memory and/or RAM. The memory 230 may include various memories such as, for example L1, L2, or L3 cache or system memory. As such, the memory 230 may include static random access memory (SRAM), dynamic RAM (DRAM), flash memory, read only memory (ROM), or other similar memory devices. The memory can store, among other things, an operating system. The RAM is used by the processor for the temporary storage of data. According to an embodiment, an operating system may contain code which, when executed by the processor, controls operation of one or more components of system 200. It will be apparent that, in embodiments where the processor implements one or more of the functions described herein in hardware, the software described as corresponding to such functionality in other embodiments may be omitted.

User interface 240 may include one or more devices for enabling communication with a user. The user interface can be any device or system that allows information to be conveyed and/or received, and may include a display, a mouse, and/or a keyboard for receiving user commands. In some embodiments, user interface 240 may include a command line interface or graphical user interface that may be presented to a remote terminal via communication interface 250. The user interface may be located with one or more other components of the system, or may located remote from the system and in communication via a wired and/or wireless communications network.

Communication interface 250 may include one or more devices for enabling communication with other hardware devices. For example, communication interface 250 may include a network interface card (NIC) configured to communicate according to the Ethernet protocol. Additionally, communication interface 250 may implement a TCP/IP stack for communication according to the TCP/IP protocols. Various alternative or additional hardware or configurations for communication interface 250 will be apparent.

Storage 260 may include one or more machine-readable storage media such as read-only memory (ROM), random-access memory (RAM), magnetic disk storage media, optical storage media, flash-memory devices, or similar storage media. In various embodiments, storage 260 may store instructions for execution by processor 220 or data upon which processor 220 may operate. For example, storage 260 may store an operating system 261 for controlling various operations of system 200.

It will be apparent that various information described as stored in storage 260 may be additionally or alternatively stored in memory 230. In this respect, memory 230 may also be considered to constitute a storage device and storage 260 may be considered a memory. Various other arrangements will be apparent. Further, memory 230 and storage 260 may both be considered to be non-transitory machine-readable media. As used herein, the term non-transitory will be understood to exclude transitory signals but to include all forms of storage, including both volatile and non-volatile memories.

While system 200 is shown as including one of each described component, the various components may be duplicated in various embodiments. For example, processor 220 may include multiple microprocessors that are configured to independently execute the methods described herein or are configured to perform steps or subroutines of the methods described herein such that the multiple processors cooperate to achieve the functionality described herein. Further, where one or more components of system 200 is implemented in a cloud computing system, the various hardware components may belong to separate physical systems. For example, processor 220 may include a first processor in a first server and a second processor in a second server. Many other variations and configurations are possible.

According to an embodiment, storage 260 of system 200 may store one or more algorithms, modules, and/or instructions to carry out one or more functions or steps of the methods described or otherwise envisioned herein. For example, storage 260 may comprise, among other instructions or data, processing instructions 262, PAC/PVC identification algorithm 263, and/or reporting instructions 264.

According to an embodiment, processing instructions 262 direct the system to perform any of the processing or analysis steps described or otherwise envisioned herein. For example, the processing instructions can instruct the system to receive ECG data, identify windows within the ECG data, and classify the QRS complexes within the ECG data.

According to an embodiment, the PAC/PVC identification algorithm 263 is a trained machine learning algorithm that analyzes ECG data in order to generate an output, where the output is an identification of one or more of the classified QRS complexes as being falsely identified as a PAC or PVC (where “falsely” means incorrectly or inaccurately identified as being a PAC or PVC, i.e., a false positive). Thus, the PAC/PVC identification algorithm is trained to analyze the input—the classified ECG data comprising the initial identification of PACs or PVCs—in order to generate the output, namely identification of one or more of the classified QRS complexes that were incorrectly identified as a PAC or PVC. The trained PAC/PVC identification algorithm can be any model that can be trained to utilize the input to generate the output, as described or otherwise envisioned herein. For example, the trained PAC/PVC identification algorithm can be a neural network or other trained machine learning model. Thus, according to an embodiment, the ECG analysis system comprises a trained PAC/PVC identification algorithm that receives the input data and generates the outputs data as described or otherwise envisioned herein.

According to an embodiment, reporting instructions 263 direct the system to provide an output—such as ECG data and/or the generated report—to a clinician or to another device or system. The provided output can be any of the information as described or otherwise envisioned herein. The system may provide the information to a user via any mechanism, including but not limited to a visual display. The information may be communicated by wired and/or wireless communication to another device. For example, the system may communicate the information to a monitor, screen, mobile phone, computer, laptop, wearable device, and/or any other device configured to allow display and/or other communication of the information.

The methods and systems described or otherwise envisioned herein, including the PVC identification algorithm, marks a significant advancement in the field of cardiology and patient care. This machine learning algorithm is designed to accurately identify QRS complexes that have been mistakenly classified as premature ventricular contractions (PVCs). By leveraging sophisticated computational techniques and a novel machine learning algorithm, the methods and systems described or otherwise envisioned herein can process and analyze electrocardiogram (ECG) data at a speed and precision far surpassing human capabilities. This improvement not only enhances diagnostic accuracy but also reduces the time required for analysis, thereby facilitating quicker and more reliable patient assessments. The algorithm's ability to consistently outperform human analysis underscores its potential to revolutionize cardiac care and improve patient outcomes.

It should be appreciated that all combinations of the foregoing concepts and additional concepts discussed in greater detail herein (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used herein, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

Unless otherwise noted, when an element or component is said to be “connected to,” “coupled to,” or “adjacent to” another element or component, it will be understood that the element or component can be directly connected or coupled to the other element or component, or intervening elements or components may be present. That is, these and similar terms encompass cases where one or more intermediate elements or components may be employed to connect two elements or components. However, when an element or component is said to be “directly connected” to another element or component, this encompasses only cases where the two elements or components are connected to each other without any intermediate or intervening elements or components.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

It should also be understood that, unless clearly indicated to the contrary, in any methods claimed herein that include more than one step or act, the order of the steps or acts of the method is not necessarily limited to the order in which the steps or acts of the method are recited.

The above-described examples of the described subject matter can be implemented in any of numerous ways. For example, some aspects can be implemented using hardware, software or a combination thereof. When any aspect is implemented at least in part in software, the software code can be executed on any suitable processor or collection of processors, whether provided in a single device or computer or distributed among multiple devices/computers.

The present disclosure can be implemented as a system, a method, and/or a computer program product at any possible technical detail level of integration. The computer program product can include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium comprises the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, comprising an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, comprising a local area network (LAN) or a wide area network (WAN), or the connection can be made to an external computer (for example, through the Internet using an Internet Service Provider). In some examples, electronic circuitry comprising, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to examples of the disclosure. It will be understood that each block of the flowchart illustrations and/or block diagrams, and combinations of blocks in the flowchart illustrations and/or block diagrams, can be implemented by computer readable program instructions.

The computer readable program instructions can be provided to a processor of a, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture comprising instructions which implement aspects of the function/act specified in the flowchart and/or block diagram or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various examples of the present disclosure. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Other implementations are within the scope of the following claims and other claims to which the applicant can be entitled.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A method for analyzing electrocardiogram (ECG) data, comprising:
receiving ECG data for a subject for a first time period, the ECG data comprise a plurality of cardiac cycles;
identifying a first window of ECG data within the received ECG data, wherein the window is a subset of the received ECG data;
classifying each of the QRS complexes within the first window of ECG data, wherein classifying comprises classifying each QRS complex as a T-wave, a premature atrial contraction (PAC), a premature ventricular contraction (PVC), or other type of heartbeat;
identifying each of a plurality of the classified QRS complexes as being a PAC or a PVC;
analyzing, using a trained machine learning algorithm, the classified QRS complexes within the first window of ECG data to generate an output, the output comprising an identification of one or more of the classified QRS complexes as being falsely identified as a PAC or PVC;
removing, from the first window, the QRS complexes identified as being falsely classified as a PAC or PVC; and
enumerating, after said removing, a number of remaining QRS complexes identified as a PAC or PVC in the first window or in the received ECG data as a whole.

2. The method of claim 1, wherein the ECG data for the subject is identified by identifying ECG data comprising a high PAC or PVC burden.

3. The method of claim 1, wherein classifying the QRS complexes within the first window of ECG data comprises classifying each QRS complex using a clustering process.

4. The method of claim 1, wherein the first window comprises an amount of ECG data that cannot be analyzed by a human being in a medically-relevant timeframe.

5. The method of claim 1, further comprising the step of generating and providing, via a user interface, a report comprising the enumeration of the number of remaining QRS complexes identified as a PAC or PVC.

6. The method of claim 1, wherein identifying each of a plurality of the classified QRS complexes as being a PAC or PVC comprises an algorithm configured to identify PACs and PVCs.

7. The method of claim 1, further comprising training the machine learning algorithm, comprising the steps of:
receiving training data comprising ECG data with identified PACs and/or PVCs for a plurality of ECG studies for a plurality of patients;
training the machine learning algorithm, with the received training data, to identify PACs and/or PVCs in ECG data; and
storing the trained machine learning algorithm.

8. A system for analyzing electrocardiogram (ECG) data, comprising:
ECG data for a subject for a first time period, the ECG data comprise a plurality of cardiac cycles;
a machine learning algorithm trained to identify premature ventricular contractions (PVCs) in ECG data;
a processor configured to: (i) identify a first window of ECG data within the received ECG data, wherein the window is a subset of the received ECG data; (ii) classify each of the QRS complexes within the first window of ECG data, wherein classifying comprises classifying each QRS complex as a T-wave, a premature atrial contraction (PAC), a premature ventricular contraction (PVC), or other type of heartbeat; (iii) identify each of a plurality of the classified QRS complexes as being a PAC or PVC; (iv) analyze, using the trained machine learning algorithm, the classified QRS complexes within the first window of ECG data to generate an output, the output comprising an identification of one or more of the classified QRS complexes as being falsely identified as a PAC or PVC; (v) remove, from the first window, the QRS complexes identified as being falsely classified as a PAC or PVC; and (vi) enumerate, after said removing, a number of remaining QRS complexes identified as a PAC or PVC in the first window or in the received ECG data as a whole.

9. The system of claim 8, wherein the ECG data for the subject is identified by identifying ECG data comprising a high PAC or PVC burden.

10. The system of claim 8, wherein classifying the QRS complexes within the first window of ECG data comprises classifying each QRS complex using a clustering process.

11. The method of claim 8, wherein the first window comprises an amount of ECG data that cannot be analyzed by a human being in a medically-relevant timeframe.

12. The system of claim 8, further comprising the step of generating and providing, via a user interface, a report comprising the enumeration of the number of remaining QRS complexes identified as a PAC or PVC.

13. The system of claim 8, wherein identifying each of a plurality of the classified QRS complexes as being a PAC or PVC comprises an algorithm configured to identify PAC and/or PVCs.

14. The system of claim 8, wherein the processor is further configured to train the machine learning algorithm, comprising:
receiving training data comprising ECG data with identified PACs or PVCs for a plurality of ECG studies for a plurality of patients;
training the machine learning algorithm, with the received training data, to identify PACs and/or PVCs in ECG data; and
storing the trained machine learning algorithm.

15. The system of claim 8, further comprising an ECG device configured to obtain the ECG data.
